SEQUENCE LISTING (9 sequences)

SEQ ID NO: 1 - protein, 1593 aa, synthetic construct
MEFKKHFKLYKSGKQWCIAAVVTFTATLGLVAGNTTVNADTLTTGGSTPQAQVNQEKGITNTSQPASDQGKTSNTGDVRNTVPQPNNSDKTNQYQADTLTTGGSTPQAQVNQEKGITNTSQPASDQGKTSNTGDVRNTVPQPNNSGKTNQSQNGQDQKESAGNDNGSTGNNDQNKGQQTTDWQKNANNQWVYNGKTDQDLKGTQYVQLPTIPDTNVQGNTNWYFVKDGIAQSGVQQWAGTYYYFDPTTYLRVDNDYRQSQWGDWYLFGNDGRILSGVQQWAGTYYYFDPTTYLRVDDDYVTSQWGLKYMFGKDGRIATGLYKWDKNNQWYYFNPTTYLAVTNNYIQANDGHWYLFTADGTAASRVAKWAGTYYYFDPQTHLRVDDNYVQSQWGDWYMFGKDGRIATGLYKWDKNNQWYYFDPTTYLAVTNNYIQANDGHWYLFTADGTAASRVAKWAGTYYYFDPVTHLRVDNNYVQSQWGDWYLFGNDGRILSGVQQWAGTYYYFDPTTYLRVDDDYVTSQWGLKYMFGKDGRIATGLYKWDKNNQWYYFDPTTYLAVTNNYIQANDGHWYLFTADGTAASRVAKWAGTYYYFDPQTHLRVDDNYVQSQWGDWYMFGKDGRIATGLYKWDKNNQWYYFDPVTYLKVTNKWVDGNYYDEDGAQAISKLVTINNRLYYFDDQGKEISNQFRTIHGDKYYFGNDSAAVTGQQTIDGKVYKFSNYGYLLGNRYGKIENGKLNIYSLADHSLIKTVEAGPWENMAYSMDSNSINNIDGYISYTGWYRPYGTSQDGKTWYPTTVADWRPILMYVWPSKDVQVKFIQYFVNHGYENSNYGLTAGSVKDLSENTASINLNEVAQNLRYVIEQHIVAAKSTSQLANDINNFITTIPELSASSELPDESGSGQVIFVNNDNTSYADSKYRLMNRTVNNQTGNDNSDYCPEFVVGNDIDNSNPVVQAENLNWEYFLLNYGKLMGYNQDGNFDGFRIDAADDMDADVLDQIGQLMNDMYHMKGNPQNANNHLSYNEGYGPGAARMLNKKGNPQLFMDARECNTLENVLGRANNRDTISHLVTDSIVNRQNDVTENEATPNWSYVTNHDIRNNLINGLIIKDHPGMGSAYKAEYANQAWQEFYADQKKTDKQYAQYNVPAQYAILLSNKDTVPQIYYGDLYNETAQYMQEKSIYYDAITTLMKARKQFVSGGQTMTKLSDNLIASVRYGKGVANANSEGTDSLSRTSGMAVIVGNNPQMAEQTISINMGRAHANEQYRNLLDTTDNGLTYNADGAENPETLTTDDNGILKVTVKGYSNPYVSGYLGVWVPVASGNQDVTTNAATVSADSNKIFESNAALDSHMIYEDFSMYQPKPTSTENHAYNIIAQNAELFNNLGITDFWMAPAYTQAGTSRYNEGYSVADRYNLGTNANPTKYGSGEELANAIAALHSAGLKVQEDIVMNQMIGLPGQEAVTVTRADNRGMQTYVNGKTYANQMYFAYTTGGGNGQETYGGKYLSELQSKYPDLFTTRAISTGVAPDPTTHITKWSAKYENGTSLQNIGIGLAVKLANGDYAYLNDSNNKAFNTTLPETMSSTDYYANIEDN

SEQ ID NO: 2 - protein, 978 aa, synthetic construct
MFGKDGRIATGLYKWDKNNQWYYFDPVTYLKVTNKWVDGNYYDEDGAQAISKLVTINNRLYYFDDQGKEISNQFRTIHGDKYYFGNDSAAVTGQQTIDGKVYKFSNYGYLLGNRYGKIENGKLNIYSLADHSLIKTVEAGPWENMAYSMDSNSINNIDGYISYTGWYRPYGTSQDGKTWYPTTVADWRPILMYVWPSKDVQVKFIQYFVNHGYENSNYGLTAGSVKDLSENTASINLNEVAQNLRYVIEQHIVAAKSTSQLANDINNFITTIPELSASSELPDESGSGQVIFVNNDNTSYADSKYRLMNRTVNNQTGNDNSDYCPEFVVGNDIDNSNPVVQAENLNWEYFLLNYGKLMGYNQDGNFDGFRIDAADDMDADVLDQIGQLMNDMYHMKGNPQNANNHLSYNEGYGPGAARMLNKKGNPQLFMDARECNTLENVLGRANNRDTISHLVTDSIVNRQNDVTENEATPNWSYVTNHDIRNNLINGLIIKDHPGMGSAYKAEYANQAWQEFYADQKKTDKQYAQYNVPAQYAILLSNKDTVPQIYYGDLYNETAQYMQEKSIYYDAITTLMKARKQFVSGGQTMTKLSDNLIASVRYGKGVANANSEGTDSLSRTSGMAVIVGNNPQMAEQTISINMGRAHANEQYRNLLDTTDNGLTYNADGAENPETLTTDDNGILKVTVKGYSNPYVSGYLGVWVPVASGNQDVTTNAATVSADSNKIFESNAALDSHMIYEDFSMYQPKPTSTENHAYNIIAQNAELFNNLGITDFWMAPAYTQAGTSRYNEGYSVADRYNLGTNANPTKYGSGEELANAIAALHSAGLKVQEDIVMNQMIGLPGQEAVTVTRADNRGMQTYVNGKTYANQMYFAYTTGGGNGQETYGGKYLSELQSKYPDLFTTRAISTGVAPDPTTHITKWSAKYENGTSLQNIGIGLAVKLANGDYAYLNDSNNKAFNTTLPETMSSTDYYANIEDN

SEQ ID NO: 3 - DNA, 2934 bp, synthetic construct
  source 1..2934: mol_type = other DNA
atgtttggtaaagatggtcggattgccacgggactatacaaatgggataagaataatcaatggtactactttgatccagtaacctacttaaaagttactaataagtgggtagacggtaattattatgatgaagatggtgcacaagccatctctaagctagtcacaattaataatcgtctttattattttgatgatcaaggaaaagaaatttcaaaccaatttagaactattcatggagataagtactattttggcaatgatagtgcggctgtaacagggcaacagacgattgatggtaaagtatacaaattcagtaattatgggtatttacttggaaatcgttatggaaagattgaaaacggtaagctgaatatttatagccttgctgaccattccttaattaagacagttgaagcaggtccatgggaaaatatggcgtatagtatggattcgaactctattaataatattgatgggtatattagttatactggttggtatcgtccatatggtacaagccaggatggtaagacttggtatccaaccacagtagctgattggcgtccaattctaatgtacgtttggcctagcaaagatgttcaagttaaatttattcagtactttgtaaatcatggttatgaaaatagtaattatggtttaacagctggttcagttaaagatttgtctgaaaacactgcttctattaacttgaatgaagttgctcaaaacttacgttatgtaattgaacaacatattgtagcagctaagagcacaagtcagcttgctaatgatataaataattttataactacaattccggaactatcggcttcatctgaattacctgatgaaagcggaagcggtcaagtaatcttcgttaataatgataatactagttacgctgatagtaagtatcgcttaatgaaccgtacggtcaataaccaaactggtaatgataatagcgattactgtcctgagtttgtggttggaaatgatattgataactctaatccggtggtacaagctgagaaccttaactgggaatacttcttgttaaattatggtaagctgatgggttataaccaggatggtaattttgatggtttccggattgatgctgctgatgatatggatgctgatgtgctcgaccagatcggacaattaatgaatgatatgtaccatatgaagggtaacccgcaaaatgctaataatcacttaagctataacgaaggatacggtccaggtgctgcccggatgttgaacaagaagggtaatccacaattgttcatggatgctagagaatgcaatactttagagaatgtcctcggacgtgctaacaatcgtgatactattagtcatttggttactgatagtattgttaaccggcaaaatgacgttacggaaaatgaagctactccaaactggtcatatgtcactaaccatgatatacgcaataacttgattaatgggttaattattaaagaccatcctggcatgggatcagcatataaagcagaatatgctaaccaagcttggcaagagttttatgctgatcaaaagaagaccgataagcagtatgcccagtataacgttccagcacaatatgcaattcttctaagcaataaagatactgtaccgcagatttactatggtgatctttacaacgaaactgcacaatatatgcaggaaaagtcgatctactatgatgcaattacgacattaatgaaggcacggaaacaatttgttagcggcggacaaacaatgaccaagcttagcgataacttaattgcaagcgttcgttatggtaagggcgttgctaatgcaaatagtgagggtactgatagtttgagtcgaacgagtggaatggcagttatcgttggtaacaatccacaaatggcagagcaaacaatttctattaatatgggtcgtgcccatgctaatgagcagtatcgtaatttgcttgacacaacggataatggtttaacatataatgctgatggtgctgaaaatccagagaccttaacaactgatgataatggtattttgaaggttactgttaagggatacagcaatccttatgtaagtggataccttggtgtgtgggtcccagttgcttctggaaatcaggatgttactactaatgccgccactgtttcagcagatagtaataagatcttcgaatctaatgctgcacttgattctcatatgatctatgaagactttagtatgtatcaacctaaaccaacaagtacagagaatcatgcatataatattattgcccaaaacgctgagctctttaacaacctgggaatcactgacttctggatggcccctgcttacacacaggctggtacgagtcgctataacgaaggttattctgtagctgatcgctataacctagggactaatgctaatccaaccaagtatggaagtggtgaagaattagcaaacgcgattgccgctttgcatagcgctggcttaaaggttcaggaagatattgttatgaaccagatgattggccttcccggtcaagaagcagtaacggttaccagagctgacaatcgtggaatgcagacttacgtcaatggtaagacatatgctaatcaaatgtactttgcatacacaaccggtggtggaaacggacaggaaacttatggtggtaagtatctgtcagagttacagtccaagtatcctgacttgttcaccacaagggccatttctaccggcgtagctcctgatccaactactcatattactaagtggtctgctaaatacgaaaacggtacatcacttcaaaatattggaataggcttagctgttaaattagctaatggcgattatgcctacttaaatgatagtaataataaagcatttaacaccactttgccagaaacgatgtcttccactgattattatgctaatattgaagacaat

SEQ ID NO: 4 - DNA, 27 bp, synthetic construct
  source 1..27: mol_type = other DNA
tgcttacacaccggctggtacgagtcg

SEQ ID NO: 5 - DNA, 25 bp, synthetic construct
  source 1..25: mol_type = other DNA
tcgtaccagccggtgtgtaagcagg

SEQ ID NO: 6 - DNA, 27 bp, synthetic construct
  source 1..27: mol_type = other DNA
tgcttacacatcggctggtacgagtcg

SEQ ID NO: 7 - DNA, 27 bp, synthetic construct
  source 1..27: mol_type = other DNA
actcgtaccagccgatgtgtaagcagg

SEQ ID NO: 8 - DNA, 26 bp, synthetic construct
  source 1..26: mol_type = other DNA
gcttacacannkgctggtacgagtcg

SEQ ID NO: 9 - DNA, 26 bp, synthetic construct
  source 1..26: mol_type = other DNA
cgactcgtaccagcmnntgtgtaagc

(19) United States
(12) Patent Application Publication
(10) Pub. No.: US 2026/0347802 A1
(43) Pub. Date: Nov. 5, 2026

(54) 4,3-alpha- glucanotransferase mutant for modulating product specificity

(71) Applicant: Jiangnan University, Wuxi (CN)

(72) Inventors: Yuxiang BAI, Wuxi (CN); Xiaoxiao LI, Wuxi (CN); Zhengyu JIN, Wuxi (CN); Ying CHEN, Wuxi (CN)

(21) Appl. No.: 19/688,751

(22) Filed: May 27, 2026

Related U.S. Application Data
(63) Continuation of application No. PCT/CN2026/073859, filed on Jan. 21, 2026.

(30) Foreign Application Priority Data
Jul. 16, 2025 (CN) 2025109810036

(51) Int. Cl. C12N 9/10

(52) U.S. Cl. CPC C12N 9/1051

(57) ABSTRACT

The present disclosure belongs to the technical fields of enzyme engineering and starch modification, and provides a 4,3-alpha-glucanotransferase mutant for modulating product specificity. The method identifies key amino acid residues through substrate channel and conservation analysis, and mutates glutamine at position 782 of Lf2970 GtfB 4,3-α-glucanotransferase-ΔN into proline, serine, aspartic acid, asparagine, leucine, phenylalanine, or tryptophan, thereby modulating the product specificity, including the proportion of glycosidic linkages and molecular weight by altering the interaction between the enzyme and the substrate. The mutants obtained in the present disclosure can be used to prepare α-glucan with diverse glycosidic linkages and molecular weight distributions, offering an effective enzymatic tool for the preparation and structural control of functional α-glucan.

### REFERENCE TO SEQUENCE LISTING

The instant application contains a Sequence Listing in XML format as a file named “PC260011A.xml”, created on 2026 May 15, of 14,307 bytes in size, and which is hereby incorporated by reference in its entirety.

### TECHNICAL FIELD

The present disclosure relates to the technical fields of enzyme engineering and starch modification, particularly to a 4,3-α-glucanotransferase mutant for modulating product specificity.

### BACKGROUND

Starch is a widely available and inexpensive carbohydrate extensively found in cereals, legumes, roots, and tubers. As an indispensable energy source in the human diet, starch is degraded by various amylases in the human digestive tract (hydrolyzing glycosidic linkages (α1→4)) to generate glucose and provide calories. However, excessive intake of calories derived from starch has led to a series of health problems, including obesity, diabetes, and cardiovascular and cerebrovascular diseases. With improved living conditions and enhanced health awareness, obtaining calories from starch is no longer a dietary necessity. The enzymatic conversion method aims at regulating the starch molecular structures and converting them into low-calorie products, which has become a major research hotspot domestically and internationally. GtfB-type 4,3/4,6-α-glucanotransferase (4,3/4,6-α-GTase) belongs to glycoside hydrolase family 70 (GH70). It synthesizes α-glucan through hydrolysis and transglycosylation using widely available and inexpensive starch or maltodextrin as substrates. Depending on the reaction specificity of the enzyme, products with different molecular structures can be obtained, including variations in molecular size, arrangement of glycosidic linkages, and degree of branching. These factors determine their respective physicochemical and biological properties, thereby defining their scopes of application. For example, 4,6-α-glucanotransferases represented by Limosilactobacillus reuteri 121 GtfB (Lr121 GtfB) catalyze starch substrates to synthesize isomalto/malto-polysaccharides (IMMPs) containing consecutive linkages (α1→6). These products can resist hydrolysis by digestive enzymes, reach the colon to serve as a carbon source for the colonic microbiota, and function as a slowly fermentable dietary fiber. A reuteran structure obtained from the enzymatic hydrolysis of starch by Lr2613 GtfB contains both (α1→4,6) branching structures and alternating linkages (α1→4)/(α1→6). These structures can limit the continuous attack of digestive enzymes on highly susceptible hydrolysable (α1→4) linkages in vivo, resulting in high levels of slowly digestible starch (SDS) and resistant starch (RS). They function as a type of carbohydrate with slowly released energy. Subsequently, a GtfB enzyme from Lactobacillus fermentum 2970 (Lf2970 GtfB) with unique catalytic specificity exhibiting 4,3-α-glucanotransferase activity was discovered and characterized. Its product is an α-glucan formed by maltooligosaccharide fragments linked via linkages (α1→3) in linear chains or branching directions, representing a first reported novel structure within the family GH70. Alternansucrase products derived from the family GH70 feature a backbone structure characterized by alternating linkages (α1→6)/(α1→3), conferring low viscosity, high solubility, and anti-digestive properties, and thus can serve as low-calorie food additives to replace fats or oils. In the medical field, polysaccharides with alternating glycosidic linkages also show potential applications. Studies have shown that polysaccharides with alternating glycosidic linkages can promote the proliferation and differentiation of human mesenchymal stem cells. In addition, studies have indicated that the presence of linkages (α1→3) in glucan can confer the capability to adsorb heavy metal ions, or make them serve as an excellent carrier for packaging and biomaterial applications. However, the mechanism underlying the synthesis of linkages (α1→3) in the products of Lf2970 GtfB remains unclear, and the structure-activity relationship of 4,3-α-glucanotransferases requires further elucidation. Therefore, understanding the mechanism for product specificity of 4,3/4,6-α-GTase is of great significance for the development of structurally tailored α-glucan products.

Existing enzyme engineering approaches for modifying GtfB-type 4,3/4,6-α-glucanotransferases primarily target an active center where chemical reactions occur. According to the α-retaining double-displacement catalytic mechanism of the family GH70, amino acid residues near the acceptor subsite are generally considered to play an important role in the transglycosylation half-reaction. Transglycosylation activity resulting from random mutagenesis of N1019 at the +1 subsite of Lr121 GtfB is affected, resulting in the synthesis of a lower proportion of linkages (α1→6) in the product. In contrast, the mutant N1019D shows increases in enzyme activity and catalytic efficiency to 420% and 590% of the wild type, respectively. In Lf2970 GtfB 4,3-α-glucanotransferase (4,3-α-GTase, 4,3-alpha-glucanotransferase), residues D991 and G1028 near the acceptor subsite of the active center contribute significantly to transglycosylation and product specificity. Mutants can introduce a significant proportion of linkages (α1→6) into the product, whereas only trace amounts of such linkages are observed in the wild type.

The substrate channel connects the active enzyme center to the surface region, affecting the entry of substrate and/or product release, thereby indirectly affecting enzyme activity, substrate specificity, and stereoselectivity. For example, mutating key amino acids in the channel of chitinase B (a glycoside hydrolase) increases the conformational flexibility of the channel wall, which impairs substrate binding. Mutations introduced into the channel of aldehyde deformylating oxygenase narrow the conformation, enhancing enzyme specificity toward short-chain aldehydes. No studies on the substrate channel of 4,3/4,6-α-glucanotransferases have been reported to date. Therefore, based on the resolved crystal structure, the present disclosure constructs a substrate channel of Lf2970 GtfB and further investigates the contribution of key amino acids within the channel to product linkage specificity, thereby enabling the preparation of α-glucan exhibiting multi-linkage specificity.

### SUMMARY

To address the above problems, the present disclosure provides a 4,3-α-glucanotransferase mutant for modulating product specificity. The 4,3-α-glucanotransferase mutant is derived from Lactobacillus fermentum (Lf) 2970, and an amino acid sequence thereof includes an amino acid sequence obtained by mutating an amino acid at position 782 of a parent amino acid sequence based on the parent Lf2970 GtfB 4,3-α-glucanotransferase-ΔN; and an amino acid sequence of the parent Lf2970 GtfB 4,3-α-glucanotransferase-ΔN is shown in SEQ ID NO. 2, or any sequence containing the amino acid sequence of the parent Lf2970 GtfB 4,3-α-glucanotransferase-ΔN shown in SEQ ID NO. 2.

In one embodiment of the present disclosure, any sequence containing the amino acid sequence of the parent Lf2970 GtfB 4,3-α-glucanotransferase-ΔN shown in SEQ ID NO. 2 may be a GtfB-type 4,3-α-glucanotransferase having the amino acid sequence shown in SEQ ID NO. 1.

In one embodiment of the present disclosure, the 4,3-α-glucanotransferase mutant may be obtained by mutating an amino acid at position 1397 of the GtfB-type 4,3-α-glucanotransferase having the amino acid sequence shown in SEQ ID NO. 1 (corresponding to position 782 of Lf2970 GtfB 4,3-α-glucanotransferase-ΔN shown in SEQ ID NO. 2). The parent Lf2970 GtfB 4,3-α-glucanotransferase-ΔN shown in SEQ ID NO. 2 is obtained by truncating 615 amino acid residues at an N-terminus of the GtfB-type 4,3-α-glucanotransferase having the amino acid sequence shown in SEQ ID NO. 1. Specifically, when the truncated amino acid residues are expressed in E. coli BL21, the catalytic activity thereof shows no significant difference compared with that of an untruncated one.

GtfB-type 4,3-α-glucanotransferase

(SEQ ID NO. 1):
MEFKKHFKLYKSGKQWCIAAVVTFTATLGLVAGNTTVNADTLTTGGSTPQ
AQVNQEKGITNTSQPASDQGKTSNTGDVRNTVPQPNNSDKTNQYQADTLT
TGGSTPQAQVNQEKGITNTSQPASDQGKTSNTGDVRNTVPQPNNSGKTNQ
SQNGQDQKESAGNDNGSTGNNDQNKGQQTTDWQKNANNQWVYNGKTDQDL
KGTQYVQLPTIPDTNVQGNTNWYFVKDGIAQSGVQQWAGTYYYFDPTTYL
RVDNDYRQSQWGDWYLFGNDGRILSGVQQWAGTYYYFDPTTYLRVDDDYV
TSQWGLKYMFGKDGRIATGLYKWDKNNQWYYFNPTTYLAVTNNYIQANDG
HWYLFTADGTAASRVAKWAGTYYYFDPQTHLRVDDNYVQSQWGDWYMFGK
DGRIATGLYKWDKNNQWYYFDPTTYLAVTNNYIQANDGHWYLFTADGTAA
SRVAKWAGTYYYFDPVTHLRVDNNYVQSQWGDWYLFGNDGRILSGVQQWA
GTYYYFDPTTYLRVDDDYVTSQWGLKYMFGKDGRIATGLYKWDKNNQWYY
FDPTTYLAVTNNYIQANDGHWYLFTADGTAASRVAKWAGTYYYFDPQTHL
RVDDNYVQSQWGDWYMFGKDGRIATGLYKWDKNNQWYYFDPVTYLKVTNK
WVDGNYYDEDGAQAISKLVTINNRLYYFDDQGKEISNQFRTIHGDKYYFG
NDSAAVTGQQTIDGKVYKFSNYGYLLGNRYGKIENGKLNIYSLADHSLIK
TVEAGPWENMAYSMDSNSINNIDGYISYTGWYRPYGTSQDGKTWYPTTVA
DWRPILMYVWPSKDVQVKFIQYFVNHGYENSNYGLTAGSVKDLSENTASI
NLNEVAQNLRYVIEQHIVAAKSTSQLANDINNFITTIPELSASSELPDES
GSGQVIFVNNDNTSYADSKYRLMNRTVNNQTGNDNSDYCPEFVVGNDIDN
SNPVVQAENLNWEYFLLNYGKLMGYNQDGNFDGFRIDAADDMDADVLDQI
GQLMNDMYHMKGNPQNANNHLSYNEGYGPGAARMLNKKGNPQLFMDAREC
NTLENVLGRANNRDTISHLVTDSIVNRQNDVTENEATPNWSYVTNHDIRN
NLINGLIIKDHPGMGSAYKAEYANQAWQEFYADQKKTDKQYAQYNVPAQY
AILLSNKDTVPQIYYGDLYNETAQYMQEKSIYYDAITTLMKARKQFVSGG
QTMTKLSDNLIASVRYGKGVANANSEGTDSLSRTSGMAVIVGNNPQMAEQ
TISINMGRAHANEQYRNLLDTTDNGLTYNADGAENPETLTTDDNGILKVT
VKGYSNPYVSGYLGVWVPVASGNQDVTTNAATVSADSNKIFESNAALDSH
MIYEDFSMYQPKPTSTENHAYNIIAQNAELFNNLGITDFWMAPAYTQAGT
SRYNEGYSVADRYNLGTNANPTKYGSGEELANAIAALHSAGLKVQEDIVM
NQMIGLPGQEAVTVTRADNRGMQTYVNGKTYANQMYFAYTTGGGNGQETY
GGKYLSELQSKYPDLFTTRAISTGVAPDPTTHITKWSAKYENGTSLQNIG
IGLAVKLANGDYAYLNDSNNKAFNTTLPETMSSTDYYANIEDN

The amino acid sequence of the parent Lf2970 GtfB 4,3-α-glucanotransferase-ΔN

(SEQ ID NO. 2):
MFGKDGRIATGLYKWDKNNQWYYFDPVTYLKVTNKWVDGNYYDEDGAQAI
SKLVTINNRLYYFDDQGKEISNQFRTIHGDKYYFGNDSAAVTGQQTIDGK
VYKFSNYGYLLGNRYGKIENGKLNIYSLADHSLIKTVEAGPWENMAYSMD
SNSINNIDGYISYTGWYRPYGTSQDGKTWYPTTVADWRPILMYVWPSKDV
QVKFIQYFVNHGYENSNYGLTAGSVKDLSENTASINLNEVAQNLRYVIEQ
HIVAAKSTSQLANDINNFITTIPELSASSELPDESGSGQVIFVNNDNTSY
ADSKYRLMNRTVNNQTGNDNSDYCPEFVVGNDIDNSNPVVQAENLNWEYF
LLNYGKLMGYNQDGNFDGFRIDAADDMDADVLDQIGQLMNDMYHMKGNPQ
NANNHLSYNEGYGPGAARMLNKKGNPQLFMDARECNTLENVLGRANNRDT
ISHLVTDSIVNRQNDVTENEATPNWSYVTNHDIRNNLINGLIIKDHPGMG
SAYKAEYANQAWQEFYADQKKTDKQYAQYNVPAQYAILLSNKDTVPQIYY
GDLYNETAQYMQEKSIYYDAITTLMKARKQFVSGGQTMTKLSDNLIASVR
YGKGVANANSEGTDSLSRTSGMAVIVGNNPQMAEQTISINMGRAHANEQY
RNLLDTTDNGLTYNADGAENPETLTTDDNGILKVTVKGYSNPYVSGYLGV
WVPVASGNQDVTTNAATVSADSNKIFESNAALDSHMIYEDFSMYQPKPTS
TENHAYNIIAQNAELFNNLGITDFWMAPAYTQAGTSRYNEGYSVADRYNL
GTNANPTKYGSGEELANAIAALHSAGLKVQEDIVMNQMIGLPGQEAVTVT
RADNRGMQTYVNGKTYANQMYFAYTTGGGNGQETYGGKYLSELQSKYPDL
FTTRAISTGVAPDPTTHITKWSAKYENGTSLQNIGIGLAVKLANGDYAYL
NDSNNKAFNTTLPETMSSTDYYANIEDN

A nucleotide sequence encoding the parent Lf2970 GtfB 4,3-α-glucanotransferase-ΔN

(SEQ ID NO. 3):
ATGTTTGGTAAAGATGGTCGGATTGCCACGGGACTATACAAATGGGATAA
GAATAATCAATGGTACTACTTTGATCCAGTAACCTACTTAAAAGTTACTA
ATAAGTGGGTAGACGGTAATTATTATGATGAAGATGGTGCACAAGCCATC
TCTAAGCTAGTCACAATTAATAATCGTCTTTATTATTTTGATGATCAAGG
AAAAGAAATTTCAAACCAATTTAGAACTATTCATGGAGATAAGTACTATT
TTGGCAATGATAGTGCGGCTGTAACAGGGCAACAGACGATTGATGGTAAA
GTATACAAATTCAGTAATTATGGGTATTTACTTGGAAATCGTTATGGAAA
GATTGAAAACGGTAAGCTGAATATTTATAGCCTTGCTGACCATTCCTTAA
TTAAGACAGTTGAAGCAGGTCCATGGGAAAATATGGCGTATAGTATGGAT
TCGAACTCTATTAATAATATTGATGGGTATATTAGTTATACTGGTTGGTA
TCGTCCATATGGTACAAGCCAGGATGGTAAGACTTGGTATCCAACCACAG
TAGCTGATTGGCGTCCAATTCTAATGTACGTTTGGCCTAGCAAAGATGTT
CAAGTTAAATTTATTCAGTACTTTGTAAATCATGGTTATGAAAATAGTAA
TTATGGTTTAACAGCTGGTTCAGTTAAAGATTTGTCTGAAAACACTGCTT
CTATTAACTTGAATGAAGTTGCTCAAAACTTACGTTATGTAATTGAACAA
CATATTGTAGCAGCTAAGAGCACAAGTCAGCTTGCTAATGATATAAATAA
TTTTATAACTACAATTCCGGAACTATCGGCTTCATCTGAATTACCTGATG
AAAGCGGAAGCGGTCAAGTAATCTTCGTTAATAATGATAATACTAGTTAC
GCTGATAGTAAGTATCGCTTAATGAACCGTACGGTCAATAACCAAACTGG
TAATGATAATAGCGATTACTGTCCTGAGTTTGTGGTTGGAAATGATATTG
ATAACTCTAATCCGGTGGTACAAGCTGAGAACCTTAACTGGGAATACTTC
TTGTTAAATTATGGTAAGCTGATGGGTTATAACCAGGATGGTAATTTTGA
TGGTTTCCGGATTGATGCTGCTGATGATATGGATGCTGATGTGCTCGACC
AGATCGGACAATTAATGAATGATATGTACCATATGAAGGGTAACCCGCAA
AATGCTAATAATCACTTAAGCTATAACGAAGGATACGGTCCAGGTGCTGC
CCGGATGTTGAACAAGAAGGGTAATCCACAATTGTTCATGGATGCTAGAG
AATGCAATACTTTAGAGAATGTCCTCGGACGTGCTAACAATCGTGATACT
ATTAGTCATTTGGTTACTGATAGTATTGTTAACCGGCAAAATGACGTTAC
GGAAAATGAAGCTACTCCAAACTGGTCATATGTCACTAACCATGATATAC
GCAATAACTTGATTAATGGGTTAATTATTAAAGACCATCCTGGCATGGGA
TCAGCATATAAAGCAGAATATGCTAACCAAGCTTGGCAAGAGTTTTATGC
TGATCAAAAGAAGACCGATAAGCAGTATGCCCAGTATAACGTTCCAGCAC
AATATGCAATTCTTCTAAGCAATAAAGATACTGTACCGCAGATTTACTAT
GGTGATCTTTACAACGAAACTGCACAATATATGCAGGAAAAGTCGATCTA
CTATGATGCAATTACGACATTAATGAAGGCACGGAAACAATTTGTTAGCG
GCGGACAAACAATGACCAAGCTTAGCGATAACTTAATTGCAAGCGTTCGT
TATGGTAAGGGCGTTGCTAATGCAAATAGTGAGGGTACTGATAGTTTGAG
TCGAACGAGTGGAATGGCAGTTATCGTTGGTAACAATCCACAAATGGCAG
AGCAAACAATTTCTATTAATATGGGTCGTGCCCATGCTAATGAGCAGTAT
CGTAATTTGCTTGACACAACGGATAATGGTTTAACATATAATGCTGATGG
TGCTGAAAATCCAGAGACCTTAACAACTGATGATAATGGTATTTTGAAGG
TTACTGTTAAGGGATACAGCAATCCTTATGTAAGTGGATACCTTGGTGTG
TGGGTCCCAGTTGCTTCTGGAAATCAGGATGTTACTACTAATGCCGCCAC
TGTTTCAGCAGATAGTAATAAGATCTTCGAATCTAATGCTGCACTTGATT
CTCATATGATCTATGAAGACTTTAGTATGTATCAACCTAAACCAACAAGT
ACAGAGAATCATGCATATAATATTATTGCCCAAAACGCTGAGCTCTTTAA
CAACCTGGGAATCACTGACTTCTGGATGGCCCCTGCTTACACACAGGCTG
GTACGAGTCGCTATAACGAAGGTTATTCTGTAGCTGATCGCTATAACCTA
GGGACTAATGCTAATCCAACCAAGTATGGAAGTGGTGAAGAATTAGCAAA
CGCGATTGCCGCTTTGCATAGCGCTGGCTTAAAGGTTCAGGAAGATATTG
TTATGAACCAGATGATTGGCCTTCCCGGTCAAGAAGCAGTAACGGTTACC
AGAGCTGACAATCGTGGAATGCAGACTTACGTCAATGGTAAGACATATGC
TAATCAAATGTACTTTGCATACACAACCGGTGGTGGAAACGGACAGGAAA
CTTATGGTGGTAAGTATCTGTCAGAGTTACAGTCCAAGTATCCTGACTTG
TTCACCACAAGGGCCATTTCTACCGGCGTAGCTCCTGATCCAACTACTCA
TATTACTAAGTGGTCTGCTAAATACGAAAACGGTACATCACTTCAAAATA
TTGGAATAGGCTTAGCTGTTAAATTAGCTAATGGCGATTATGCCTACTTA
AATGATAGTAATAATAAAGCATTTAACACCACTTTGCCAGAAACGATGTC
TTCCACTGATTATTATGCTAATATTGAAGACAAT

In one embodiment of the present disclosure, the mutant is:
obtained by mutating glutamine at position 782 of the 4,3-α-glucanotransferase having the amino acid sequence shown in SEQ ID NO. 2 into proline, designated as Q782P; or
obtained by mutating the glutamine at position 782 of the 4,3-α-glucanotransferase having the amino acid sequence shown in SEQ ID NO. 2 into serine, designated as Q782S; or
obtained by mutating the glutamine at position 782 of the 4,3-α-glucanotransferase having the amino acid sequence shown in SEQ ID NO. 2 into aspartic acid, asparagine, leucine, phenylalanine, or tryptophan, designated as Q782D, Q782N, Q782L, Q782F, and Q782W, respectively.

In one embodiment of the present disclosure, the 4,3-α-glucanotransferase mutant may also be obtained by mutating an amino acid at position 1397 of the GtfB-type 4,3-α-glucanotransferase having the amino acid sequence shown in SEQ ID NO. 1 (corresponding to position 782 of the parent Lf2970 GtfB 4,3-α-glucanotransferase-ΔN shown in SEQ ID NO. 2), designated as Q1397P, Q1397S, Q1397D, Q1397N, Q1397L, Q1397F, and Q1397W, respectively.

In one embodiment of the present disclosure, a nucleotide sequence encoding the parent Lf2970 GtfB 4,3-α-glucanotransferase-ΔN is shown in SEQ ID NO. 3.

The present disclosure further provides a gene encoding the above mutant.

The present disclosure further provides a recombinant vector carrying the above gene.

In one embodiment of the present disclosure, the recombinant vector uses pET15b as an expression vector.

The present disclosure further provides the above mutant, or a recombinant cell carrying the above gene or carrying the above recombinant vector.

In one embodiment of the present disclosure, the recombinant cell uses a bacterium or fungus as an expression host.

In one embodiment of the present disclosure, the recombinant cell uses E. coli BL21 (DE3) as the expression host.

The present disclosure further provides a method for constructing a substrate channel mutant of Lf2970 GtfB 4,3-α-glucanotransferase. The mutant is obtained as following: through sequence alignment, a weakly conserved position Q782 is selected and mutated to amino acids present at corresponding positions in other enzymes, thereby yielding mutants Q782P and Q782S; or Q782 is randomly mutated to yield mutants Q1397D, Q1397N, Q1397L, Q1397F, and Q1397W, respectively.

The present disclosure further provides a method for preparing α-glucan using Lf2970 GtfB 4,3-α-glucanotransferase and the above mutants. The method includes: using starch or a starch derivative as a substrate to prepare the α-glucan through a reaction. The resulting synthesized product features different glycosidic linkage compositions and molecular weight distributions. The α-glucan contains glycosidic linkages (α1→4), glycosidic linkages (α1→3), and glycosidic linkages (α1→6).

In one embodiment of the present disclosure, a concentration of the substrate is 0.25%-30% (w/v).

In the present disclosure, w/v represents g/mL; for example, a concentration of the substrate of 2% (w/v) indicates that a concentration of the substrate is 0.02 g/mL.

In one embodiment of the present disclosure, the buffer system has a concentration of 10-50 mM, and a pH of 4-6.

In one embodiment of the present disclosure, a reaction temperature is controlled at 37-40 °C, and a reaction duration is 4-48 hours.

In one embodiment of the present disclosure, a dosage of the enzyme used in the reaction is 10-50 U/g of starch on a dry basis.

In one embodiment of the present disclosure, the α-glucan has a proportion of linkages (α1→3) of 20.6%-26.3%, a proportion of linkages (α1→6) of 1.5%-7.9%, and a weight-average molecular weight range of 38.43 kDa-1,724.68 kDa.

In one embodiment of the present disclosure, the method uses starch or a starch derivative as a substrate, where the starch includes amylopectin and amylose, and the starch derivative includes maltodextrin and maltooligosaccharides (with a degree of polymerization≥3).

In one embodiment of the present disclosure, when the amylose is used as the substrate, the starch is first dissolved in alkali, then neutralized with acid, followed by addition of a buffer solution, and the mutant or the recombinant mutant enzyme is then added for modification to obtain a synthesized α-glucan.

In one embodiment of the present disclosure, when the amylopectin is used as the substrate, a buffer solution is added to the starch, the starch is gelatinized and cooled, followed by addition of a buffer solution, and the mutant or the recombinant mutant enzyme is then added for modification to obtain a synthesized α-glucan.

In one embodiment of the present disclosure, when the dextrin is used as the substrate, a buffer solution is first added, and the mutant or the recombinant mutant enzyme is then added for modification to obtain a synthesized α-glucan.

The present disclosure further provides use of the above mutant or recombinant cell in preparing an α-glucan. The use includes: using the above mutant, or the recombinant cell, or the recombinant mutant enzyme expressed by the recombinant cell as a catalyst, and using starch or a starch derivative as a substrate to prepare the α-glucan through a reaction. The α-glucan has glycosidic linkages (α1→4), glycosidic linkages (α1→3), and glycosidic linkages (α1→6).

### Beneficial Effects

(1) The 4,3-α-glucanotransferase mutant provided in the present disclosure can effectively convert the substrate. The resulting α-glucan/oligosaccharide introduced new linkages (α1→6) and branching (α1→4,6) compared with the wild type. A proportion of the linkages (α1→6) increases from 1.5% to 7.9%, and a proportion of the linkages (α1→3) increases from 21.9% to 26.3%. Compared with the non-mutated Lf2970 GtfB 4,3-α-glucanotransferase, a total content of the linkages (α1→6) and linkages (α1→3) are increased by 48%. Through these mutations, the proportions of the linkages (α1→6) and linkages (α1→3) in the product of Lf2970 GtfB are modulated, thereby improving the product's resistance to digestion. As a novel soluble dietary fiber and prebiotic, the resulting product has great application potential in the fields of food and pharmaceuticals.
(2) Expression levels and enzyme activities of the 4,3-α-glucanotransferase mutants provided in the present disclosure are not significantly affected, thereby retaining great potential for industrial application.

### BRIEF DESCRIPTION OF THE DRAWINGS

FIG. 1 is a schematic diagram of a substrate channel of Lf2970 GtfB 4,3-α-glucanotransferase obtained through software simulation.

FIG. 2 illustrates a multiple sequence alignment of representative family GH70 enzymes in a channel region.

FIG. 3 illustrates protein purification results of a wild type and mutants, where M: protein marker; 1: cell pellet; 2: crude enzyme; 3-6: eluates obtained with imidazole at different concentration gradients.

FIG. 4A is a one-dimensional ¹H NMR spectrum of a wild-type Lf2970 GtfB.

FIG. 4B is a one-dimensional ¹H NMR spectrum of Q782P.

FIG. 4C is a one-dimensional ¹H NMR spectrum of Q782L.

FIG. 4D is a one-dimensional ¹H NMR spectrum of Q1397F.

### DETAILED DESCRIPTIONS OF THE EMBODIMENTS

The technical solutions of the present disclosure will be described below clearly and completely in conjunction with the accompanying drawings. Apparently, the described embodiments are merely some of, rather than all of, the embodiments of the present disclosure. Based on the embodiments of the present disclosure, all the other embodiments obtained by those of ordinary skill in the art without making creative efforts shall fall within the scope of protection of the present disclosure.

### DEFINITIONS OR TERMS USED IN THE PRESENT DISCLOSURE

Mutant: As used herein, when referring to the use of variants of the present disclosure, the term “mutant” or “polypeptide variant” or “polypeptide” or “RNA polymerase mutant” means a polypeptide having RNA polymerase enzyme activity and containing one or more (e.g., several) alterations (i.e., substitutions, insertions, and/or deletions) at positions relative to a “parental” RNA polymerase. Substitution means replacing an amino acid occupying a specific position with a different amino acid; deletion means removing an amino acid occupying a specific position; and insertion means adding an amino acid immediately adjacent to and following the amino acid occupying a specific position. In describing the variants of the present disclosure, the following nomenclature has been adapted for ease of reference. Accepted IUPAC one-letter or three-letter amino acid abbreviations are used. Substitution: For amino acid substitutions, the following nomenclature is used: original amino acid, position, and substituted amino acid. Therefore, substitution of the glutamine at position 782 with proline is denoted as “Q782P”.

Wild-type enzyme: When referring to an amino acid sequences or a nucleic acid sequence, the term “wild-type” means that the amino acid sequence or nucleic acid sequence is a natural or naturally occurring sequence. As used herein, the term “naturally occurring” means any substance (e.g., protein, amino acid, or nucleic acid sequence) found in nature. In contrast, the term “non-naturally occurring” means any substance not found in nature (e.g., recombinant nucleic acid and protein sequences produced in a laboratory, or modifications of wild-type sequences). When the parent enzyme is not a variant enzyme, the terms “wild-type enzyme” and “parent enzyme” may be used interchangeably.

Expression: As used herein, the term “expression” means any step involved in the production of a variant, including but not limited to transcription, post-transcriptional modification, translation, post-translational modification, and secretion.

Expression vector: As used herein, the term “expression vector” means a linear or circular DNA molecule including a polynucleotide encoding a variant, which is operably linked to control sequences providing for its expression.

Host cell: The term “host cell” means any cell type that is amenable to transformation, transfection, transduction, and the like, with a nucleic acid construct or expression vector including a polynucleotide of the present disclosure. The term “host cell” encompasses any progeny of a parental cell that differs from the parental cell due to mutations occurring during replication, as well as recombinant host cells, isolated host cells (e.g., isolated recombinant host cells), and heterologous host cells.

Recombinant: When used to refer to a cell, nucleic acids, protein, or vector, the term “recombinant” means that it has been modified from its natural state. Therefore, for example, a recombinant cell expresses a gene not found in the cell in its natural (non-recombinant) form, or expresses a natural gene at a different level or under different conditions compared to those found in nature. A recombinant nucleic acid differs from a natural sequence in that one or more nucleotides are operably linked to a heterologous sequence (e.g., a heterologous promoter in an expression vector). A recombinant protein may differ from a natural sequence in that one or more amino acids are fused to a heterologous sequence. A vector containing a nucleic acid encoding a polypeptide is a recombinant vector. The term “recombinant” is synonymous with “genetically modified,” and “transgenic”.

It should be noted that the abbreviations of amino acid residues used herein follow the general definitions in the art, however, the full names of amino acids are additionally indicated in parentheses following the abbreviations in some instances throughout the text. “Amino acid residue” and “amino acid” used herein may be used interchangeably. The amino acid sequences herein are numbered from the N-terminus.

### Detection Methods Involved in the Following Examples are as Follows:

### Enzyme Activity Determination Method:

The activities of the wild-type enzyme and mutant enzymes were determined using an amylose-iodine staining method. The reaction was conducted at 40 °C using 0.25% (w/v) amylose as a substrate, with a dosage of the enzyme of 9 μg/mL. A buffer system consisted of 25 mM NaAc and 5 mM CaCl₂) at pH 5.5. One unit (U) of enzyme activity was defined as an amount of enzyme required to catalyze the conversion of 1 mg of amylose per minute. The hydrolytic activity was determined using a 3,5-dinitrosalicylic acid (DNS) method. The reaction system and dosage of enzyme were identical to those used for total enzyme activity. One unit (U) of enzyme activity was defined as an amount of enzyme required to release 1 mg of reducing sugar from amylose per minute under optimal conditions.

### Kinetic Parameter Determination:

Total enzyme activity was measured at different amylose concentrations (0.025%, 0.03%, 0.04%, 0.05%, 0.1%, 0.15%, 0.2%, and 0.25%). Kinetic parameters Kₘ and k_{cat} were calculated according to a Michaelis-Menten equation:

$$v = \frac{{V}_{max} [ S ]}{{K}_{{m}^{+}} [ S ]} , {k}_{cat} = \frac{{V}_{max}}{[ E ]} ,$$

where Vₘₐₓ denotes a maximum reaction velocity, [S] denotes a substrate concentration, and [E] denotes an enzyme concentration. Finally, the catalytic efficiency (k_{cat}/Kₘ) of enzyme was calculated.

### Analysis of Glycosidic Linkage Composition of Products:

In the present disclosure, the products generated from the modification of starch derivatives with the wild type and mutant Lf2970 GtfB 4,3-α-glucanotransferase-ΔN were analyzed using NMR and methylation analysis to determine their types and contents of glycosidic linkage, and branching degree.

### ¹H Spectroscopy:

20 mg of a lyophilized sample was taken, 1 mL D20 was added to obtain a mixture, and the mixture was boiled for 1 hour and then immediately lyophilized. After lyophilization, 1 mL of D20 was added, boiled for 1 hour, and then immediately lyophilized for later use. Finally, the sample was dissolved in D20, boiled in a boiling water bath for 1 hour, and a one-dimensional ¹H NMR spectrum was recorded on an AVANCE-600 MHz spectrometer (Bruker, Germany), with a probe temperature of 25 °C.

### Methylation Analysis:

Lyophilized oligosaccharides/polysaccharides (60 mg) were dissolved in 20 mL DMSO, 20 mL of a NaOH solution dissolved in DMSO was then added to obtain a mixture, and the mixture was allowed to react for 1 hour; and liquid CH₃I was then added dropwise under ice bath conditions to allow for methylation reaction for 2 hours. The resulting methylated products were dialyzed using dialysis tubing with a molecular weight cutoff of 1,000 Da, followed by extraction with chloroform, drying over sodium sulfite, and concentration under a stream of nitrogen. Subsequently, the samples were hydrolyzed with trifluoroacetic acid (2 M), reduced with NaBD₄, and acetylated using acetic anhydride and pyridine (1:1, v/v). The resulting partially methylated alditol acetates (PMAAs) were analyzed using a gas chromatography-mass spectrometry (GC-MS) system (Agilent Technologies, Santa Clara, USA). The resulting EI-MS spectra were compared with those in the CCRC Spectral Database for PMAAs to determine the types and relative proportions of glycosidic linkages.

Product molecular weight analysis: Relative molecular weight distribution of the products generated from enzymatic modification of starch derivatives was determined using high-performance gel permeation chromatography (HPGFC). The lyophilized reaction sample was dissolved in purified water to a final concentration of 5 mg/mL and filtered through a 0.45 μm cellulose acetate membrane. To monitor the molecular weight distribution of the sample, a 30 μL aliquot of the sample was injected into a gel filtration chromatography system equipped with a refractive index detector.

### Example 1: Simulation of Substrate Channel and Identification of Key Residues of Lf2970 GtfB 4,3-α-Glucanotransferase-ΔN

### 1. Construction of Substrate Channel

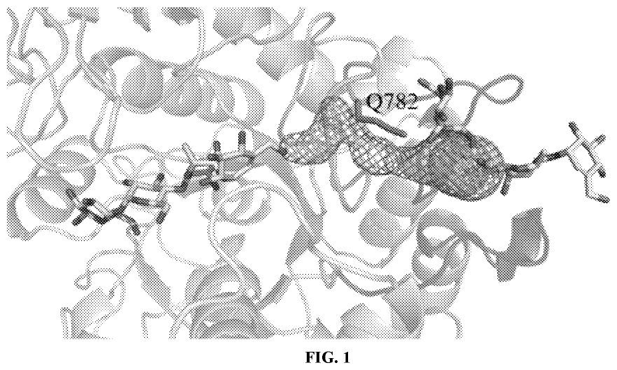

Using a crystal structure of an LrN1 GtfB-acarbose complex (PDB: 8HW3) as a template, a structural model of a Lf2970 GtfB 4,3-α-glucanotransferase-acarbose complex was established by homology modeling. In the model, two acarbose molecules were found to bind at similar positions in an active center and on a surface. A substrate channel, as shown in FIG. 1, was calculated using the CAVER Web software. The channel is located on a donor side, and two ends thereof are connected to a surface and a ligand at the active center, respectively, demonstrating the credibility of simulation.

### 2. Selection of Mutation Sites

The substrate channel, serving as a “bridge” between a surrounding solvent and the active center, has an important impact on enzyme activity, catalytic specificity, and stability. However, the role of key amino acids in the channel during the enzymatic catalysis has not yet been reported in family GH70 enzymes. Analysis of amino acids surrounding the substrate channel of Lf2970 GtfB 4,3-α-glucanotransferase-ΔN revealed that position Q782 is located in a relatively narrow region of the channel and exhibits weak conservation. Therefore, modification of the position may alter the spatial conformation of the channel, thereby affecting the orientation of the substrate in the active center, and indirectly resulting in changes in product specificity.

### Example 2: Design and Construction of Mutants at Position Q782 in the Channel of Lf2970 GtfB 4,3-α-Glucanotransferase-ΔN

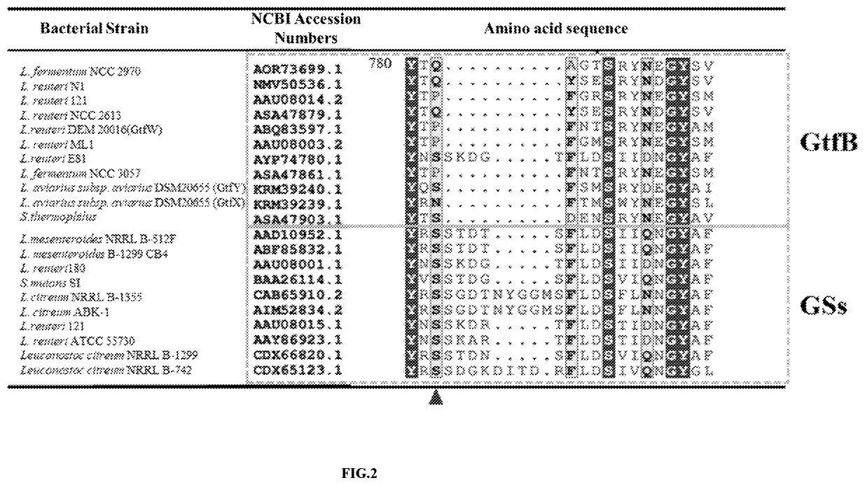

Sequence alignment was performed on glucansucrases (GSs) and GtfB enzymes, representative member of the family GH70, as shown in FIG. 2. It was observed that the glutamine at position 782 in Lf2970 GtfB 4,3-α-glucanotransferase-ΔN was substituted by proline or serine in other GH70 enzymes. Therefore, the glutamine residue at position 782 was mutated to proline and serine, respectively, to construct site-directed mutants Q782P and Q782S. In addition, to further investigate the contribution of this position, random mutagenesis was also performed. Primers for the above mutants are listed in Table 1.

TABLE 1
Primer sequences for mutants

| | Primer sequence (5′→3′) |
|---|---|
| Mutant | (SEQ ID NO. 4-9) |
| Q782P | F: TGCTTACACACCGGCTGGTACGAGTCG |
| | R: TCGTACCAGCCGGTGTGTAAGCAGG |
| Q782S | F: TGCTTACACATCGGCTGGTACGAGTCG |
| | R: ACTCGTACCAGCCGATGTGTAAGCAGG |
| Q782X | F: GCTTACACANNKGCTGGTACGAGTCG |
| | R: CGACTCGTACCAGCMNNTGTGTAAGC |

Note: Underlined sequences indicate mutated nucleotides.

Using pET15b-Lf2970 GtfB 4,3-α-glucanotransferase-ΔN as a template, site-directed mutagenesis was performed on a target amino acid using whole-plasmid PCR.

The PCR program was as follows: initial denaturation at 98 °C for 3 minutes, followed by 30 cycles (denaturation at 98 °C for 20 seconds, annealing at 55 °C for 30 seconds, and extension at 68 °C for 9 minutes), and a final extension at 68 °C for 10 minutes.

The resulting PCR fragments after reaction were digested with DpnI enzyme and then transformed into competent Escherichia coli (E. coli) DH5a cells by a heat-shock method for plasmid cloning. The successfully sequenced plasmids were then transformed into E. coli BL21 competent cells to obtain the following recombinant strains of E. coli containing the respective mutants: BL21/pET15b-Q782P-AN, BL21/pET15b-Q782S-AN, BL21/pET15b-Q782D-AN, BL21/pET15b-Q782N-AN, BL21/pET15b-Q782L-AN, BL21/pET15b-Q782F-AN, and BL21/pET15b-Q782W-AN.

In addition, a recombinant E. coli strain containing the wild-type enzyme was obtained: BL21/pET15b-Lf2970 GtfB 4,3-α-glucanotransferase-ΔN.

### Example 3: Expression and Purification of Lf2970 GtfB 4,3-α-Glucanotransferase-ΔN and Mutants

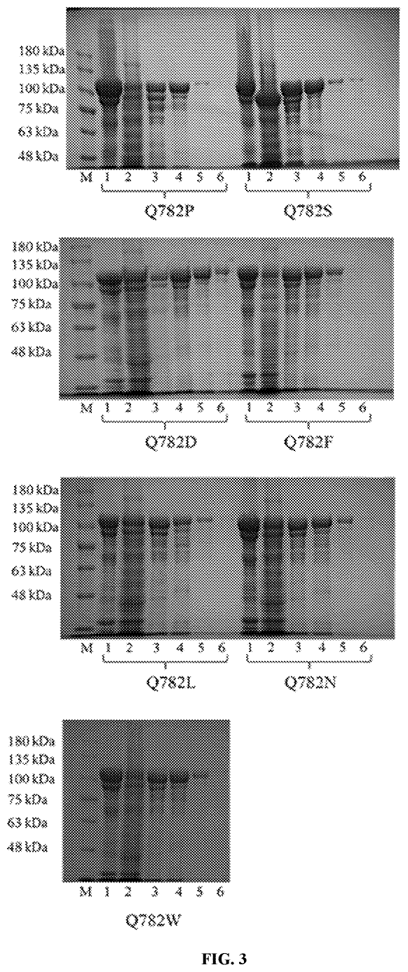

The specific steps are as follows:
(1) The recombinant E. coli strains containing the wild-type and mutant genes obtained in Example 2 were respectively inoculated into LB medium containing ampicillin (100 μg/mL), and cultured at 37 °C and 200 rpm. When OD₆₀₀ reached 0.4-0.6, the cultures were subjected to an ice bath treatment for 15 minutes, followed by the addition of isopropyl β-D-1-thiogalactopyranoside (IPTG, 0.1 mM). Enzyme expression was induced at 16 °C and 160 rpm for 20 hours, and fermentation broths were obtained, respectively.
(2) The obtained fermentation broths were centrifuged at 4 °C and 8,000×g for 30 minutes to harvest the cells. The cell pellets were then resuspended in 25 mM Tris-HCl (250 mM NaCl, pH 7.5) at a ratio of 1 g cell pellets per 10 mL buffer, and then disrupted by ultrasonication on ice for 20 minutes (30% power with disruption of 2 seconds with an interval of 3 seconds). The cell lysate after disruption was centrifuged at 4 °C and 8,000×g for 40 minutes, and the resulting supernatant was a crude enzyme solution. The following solutions were respectively prepared and obtained:
purified enzyme solution containing a wild-type Lf2970 GtfB 4,3-α-glucanotransferase-ΔN, and crude enzyme solutions containing Q782P, Q782S, Q782D, Q782N, Q782L, Q782F, and Q782W, respectively.
(3) The crude enzyme solutions obtained in the step (2) were purified using nickel affinity chromatography, elution was performed sequentially using 20 mM Tris-HCl (250 mM NaCl, pH 7.5) and 20 mM Tris-HCl (250 mM NaCl, pH 7.5) containing different concentrations of imidazole. Flow-through fractions were collected, and purity of each fraction was verified by polyacrylamide gel electrophoresis. The results are shown in FIG. 3.

The results demonstrate that both the wild-type and mutant enzymes of the present disclosure were expressed.

### (4) Enzyme Activity Determination

The enzyme activities of the purified enzyme solutions obtained in the step (3) were separately determined, and the results are shown in Table 2. Q782P and Q782S showed significantly increased total enzyme activities while retaining transglycosylation activities, and their relative hydrolytic activities showed no significant change compared with the wild type. The total enzyme activity of Q782W was also higher than that of the wild type; however, its hydrolytic activity increased significantly, resulting in an increased relative hydrolytic activity and strong hydrolytic property. Q782N, Q782F, and Q782D also showed strong hydrolytic properties. Specifically, Q782F and Q782D showed a significant decrease in total enzyme activity. The hydrolytic activity of Q782L decreased significantly to 0.29 U/mg, resulting in reduced relative hydrolytic activity and indicating relatively enhanced transglycosylation activity.

TABLE 2
Enzyme activities of wild-type Lf2970 GtfB 4,3-α-glucanotransferase-
ΔN and mutants thereof^{a}.

| | Enzyme activity (U/mg) | | |
|---|---|---|---|
| | Total | Hydrolytic | |
| Enzyme | enzyme activity | activity | Relative hydrolytic activity^{b} |
| WT | 12.94 ± 0.24^{c} | 1.67 ± 0.15^{d} | 0.13 ± 0.01^{cd} |
| Q782P | 25.61 ± 0.24^{a} | 2.78 ± 0.16^{c} | 0.11 ± 0.01^{d} |
| Q782S | 24.84 ± 1.34^{a} | 3.62 ± 0.01^{b} | 0.15 ± 0.01^{c} |
| Q782D | 5.62 ± 1.49^{f} | 1.01 ± 0.12^{e} | 0.18 ± 0.03^{b} |
| Q782N | 13.72 ± 0.40^{c} | 2.97 ± 0.14^{c} | 0.22 ± 0.01^{a} |
| Q782L | 7.45 ± 0.16^{e} | 0.29 ± 0.01^{f} | 0.04 ± 0.01^{e} |
| Q782F | 10.67 ± 0.63^{d} | 1.68 ± 0.02^{d} | 0.16 ± 0.01^{bc} |
| Q782W | 20.78 ± 0.31^{b} | 4.77 ± 0.06^{a} | 0.23 ± 0.06^{a} |

a. Each value represents the mean±standard deviation of three independent replicate experiments. Different lowercase letters within the same column indicate statistically significant differences (p<0.05).
b.

$$Relative hydrolytic activity = \frac{Hyrolytic activity}{Total enzyme activity} .$$

### Example 4: Preparation of α-Glucan Using Wild Type and Mutant Lf2970 GtfB 4,3-α-Glucanotransferase-ΔN

The specific steps are as follows:
(1) Debranching treatment of potato starch to obtain amylose. Specifically:
Potato starch was dissolved in a buffer solution (20 mM sodium acetate buffer, pH 4.6) to a final concentration of 2% (w/v), the resulting mixture was subjected to a boiling water bath for 1 hour to ensure uniform dispersion of starch. After cooling to room temperature, a commercial pullulanase (enzyme activity≥1,000 U/mL) was added at a volume 1% (v/v) of a solution volume, and the reaction was carried out at 60 °C for 24 hours, followed by a boiling water bath for 15 minutes to inactivate the enzyme. The resulting solution was then centrifuged (8000×g, 20 min), and a supernatant was collected and dried at 40 °C. The resulting product was ground, sieved, washed three times with 90% methanol, and dried again at 40 °C to obtain debranched potato starch.
(2) The debranched potato starch obtained in the step (1) was first dissolved in 1 M sodium hydroxide solution to a final starch concentration of 4% (w/v). The solution was then neutralized with an equal volume of 1 M hydrochloric acid, followed by addition of a buffer solution (25 mM NaAc, 5 mM CaCl₂), pH 5.5) to adjust the final starch concentration to 1% (w/v). After cooling to room temperature, the purified enzyme solutions of Lf2970 GtfB 4,3-α-glucanotransferase-ΔN and mutants thereof prepared in Example 2 were added at a dosage of the enzyme of 50 U/g. The reaction was carried out at 40 °C for 24 hours, and terminated by a boiling water bath for 15 minutes, and the resulting product was freeze-dried to obtain lyophilized powder.

### Example 5: Product Analysis of Wild Type and Mutant Enzymes of Lf2970 GtfB 4,3-α-Glucanotransferase-ΔN

### 1. Analysis of Product Molecular Weight and Types of Glycosidic Linkages

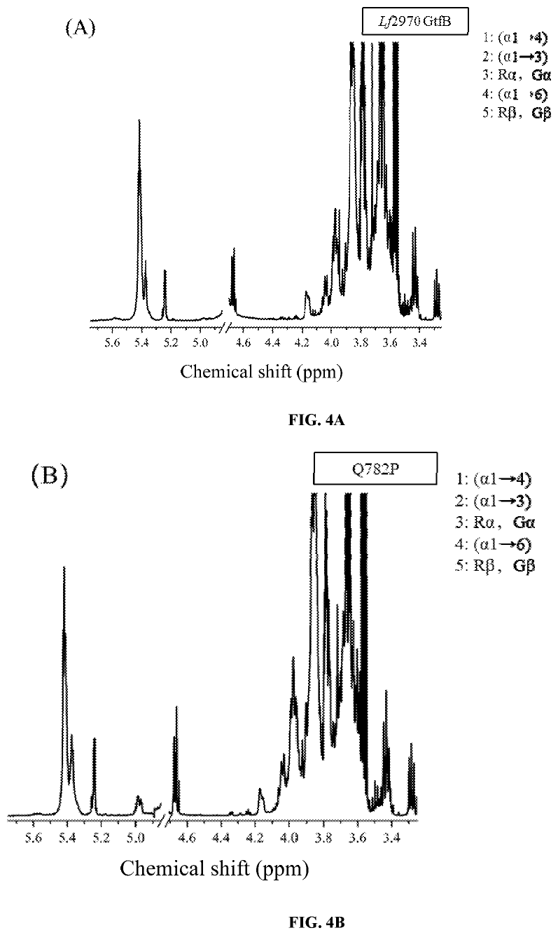

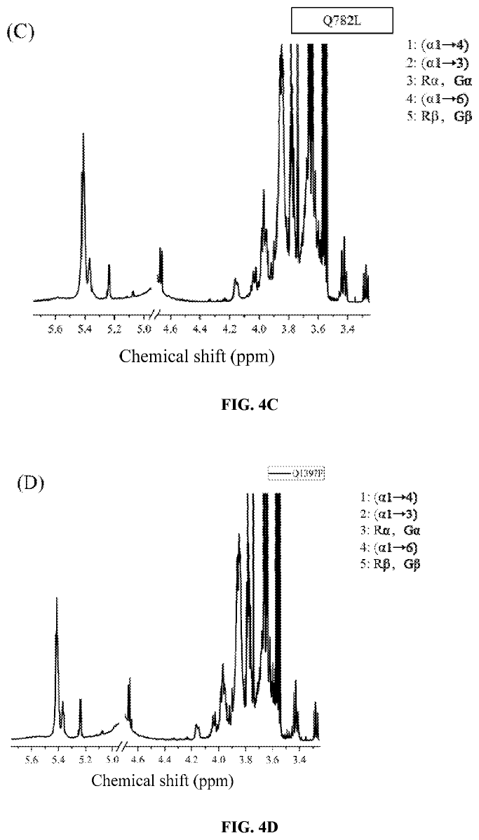

The products prepared in Example 4 were analyzed by one-dimensional ¹H NMR spectroscopy and molecular weight analysis. The results are shown in Table 3 and FIG. 4A and FIG. 4B and FIG. 4C and FIG. 4D.

TABLE 3
Glycosidic linkage composition and molecular weight
distribution of products generated by wild-type and
mutant Lf2970 GtfB 4,3-α-glucanotransferase-ΔN

| | Glycosidic | | | Molecular weight distribution, | |
|---|---|---|---|---|---|
| | linkages (%) | | | Mw (kDa) | |
| Enzyme | (α1→4) | (α1→3) | (α1→6) | Peak 1 | Peak 2 |
| Lf2970 GtfB | 76.9 | 21.9 | 1.2 | 970.67 | 0.72 |
| 4,3-α- | | | | | |
| glucano- | | | | | |
| transferase- | | | | | |
| ΔN | | | | | |
| Q782S | 76.9 | 20.8 | 2.3 | 989.67 | 1.01 |
| Q782P | 65.8 | 26.3 | 7.9 | 38.43 | 0.30 |
| Q782D | 79.4 | 20.6 | ND | 422.26 | 1.27 |
| Q782N | 75.8 | 21.1 | 3.0 | 482.61 | 1.68 |
| Q782L | 74.1 | 22.2 | 3.7 | 1724.68 | 2.48 |
| Q782F | 75.2 | 21.1 | 3.8 | 326.13 | 1.17 |
| Q782W | 79.4 | 20.6 | ND | 393.66 | 1.18 |

ND: Not detected

The results indicate that:
(1) Regarding the molecular weight distribution, both wild-type and mutant products of Lf2970 GtfB 4,3-α-glucanotransferase-ΔN exhibited bimodal distributions. Specifically, a molecular weight of the product from the Q782P mutant significantly decreased to 38.43 kDa. The molecular weights of mutants Q782D, Q782N, Q782F, and Q782W slightly decreased but remained within the same order of magnitude as that of the wild type. The molecular weight of the product from Q782S was similar to that of the wild type, whereas the product molecular weight of the product from Q782L was significantly higher than that of the wild type, demonstrating enhanced reaction continuity.
(2) Regarding glycosidic linkage composition, the Q782P mutant exhibited a significant change in product linkage specificity. A proportion of the linkages (α1→4) decreased to 65.8%, while proportions of the linkages (α1→3) and linkages (α1→6) significantly increased to 26.3% and 7.9%, respectively. Compared with the wild type, a proportion of the linkages (α1→3) increased by 20%, and the linkages (α1→6) surged by 558%, indicating strong transglycosylation activity. The non-polar mutants Q782L and Q782F both exhibited strong α1,6-transglycosylation activity. The difference was that Q782P simultaneously enhanced both α1,3-transglycosylation and α1,6-transglycosylation activity greatly, indicating that the rigidity of the position has a greater impact on the catalytic specificity. The product linkage specificity of Q782D and Q782W was similar to that of the wild type.

### 2. Methylation Analysis of Polysaccharide Products

For mutant Q782P, which exhibited significant changes in the proportion of glycosidic linkages in its products, methylation analysis was further performed to determine the types and proportions of linear/branched linkages in the polysaccharides, using the wild type as a control. The results are shown in Table 4. In the product of Q782P, proportions of linear linkages (α1→4) and branched linkages (α1→3,4) were similar to those of the wild type. However, the linear linkages (α1→3) significantly decreased from 20% to 11%, while a content of branched linkages (α1→4,6) significantly increased to 9%. Based on a combined analysis of NMR and methylation results, it can be observed that the Q782P mutation alters the product linkage specificity by attenuating the synthesis of the linkages (α1→3) and promoting the synthesis of the linkages (α1→4,6).

TABLE 4
Methylation analysis:

| | Molar ratio (%) | | | | | |
|---|---|---|---|---|---|---|
| | | →3)-α-D- | →4)-α-D-Glcp- | →6)-α-D-Glcp- | →3,4)-α-D-Glcp- | →4,6)-α-D- |
| Enzyme | α-D-Glcp-(1→ | Glcp-(1→ | (1→ | (1→ | (1→ | Glcp-(1→ |
| Lf2970 | 3 | 20 | 46 | <1 | 30 | <1 |
| GtfB | | | | | | |
| 4,3-α-glucanotransferase-ΔN | | | | | | |
| Q782P | 3 | 11 | 43 | 1 | 34 | 9 |

Although the present disclosure has been disclosed as above in the form of preferred embodiments, it is not intended to limit the present disclosure. Those skilled in the art can make various modifications and variations without departing from the spirit and scope of the present disclosure. Therefore, the scope of protection of the present disclosure should be defined by the claims.

What is claimed is:

1. A 4,3-α-glucanotransferase mutant for modulating product specificity, wherein an amino acid sequence of the 4,3-α-glucanotransferase mutant comprises an amino acid sequence obtained by mutating glutamine at position 782 of a parent amino acid sequence into proline on the basis of the parent amino acid sequence; and the parent amino acid sequence is shown in SEQ ID NO. 2, or any sequence containing the parent amino acid sequence shown in SEQ ID NO. 2.

2. A 4,3-α-glucanotransferase mutant for modulating product specificity, wherein an amino acid sequence of the 4,3-α-glucanotransferase mutant comprises an amino acid sequence obtained by mutating amino acid at position 782 of a maternal amino acid sequence into proline on the basis of the maternal amino acid sequence; wherein the maternal amino acid sequence is shown in SEQ ID NO. 2, or any sequence containing the maternal amino acid sequence shown in SEQ ID NO. 2;
the amino acid sequence of the 4,3-α-glucanotransferase mutant comprises one of the following amino acid sequences:
(1) an amino acid sequence obtained by mutating the glutamine at position 782 of the 4,3-α-glucanotransferase sequence shown in SEQ ID NO. 2 into serine; or
(2) an amino acid sequence obtained by mutating the glutamine at position 782 of the 4,3-α-glucanotransferase sequence shown in SEQ ID NO. 2 into aspartic acid, asparagine, leucine, phenylalanine, or tryptophan.

3. A method for preparing a 4,3-α-glucanotransferase mutant that regulates product specificity, wherein the method comprises the following steps:
(1) analyzing the surface binding structure of the enzyme to determine the surface binding amino acids of Lactobacillus fermentum 2970 4,3-α-glucanotransferase, and based on this, simulating the channel through which the substrate enters the enzyme active site, with key amino acid sites in the channel identified through sequence alignment;
(2) designing mutation primers for the key amino acids, using a plasmid carrying the gene encoding Lactobacillus fermentum 2970 4,3-α-glucanotransferase as a template to perform the mutation, thereby obtaining the mutated plasmid;
(3) transferring the mutated plasmid into a host strain, selecting positive single clones for fermentation culture to obtain crude enzyme of the Lactobacillus fermentum 2970 4,3-α-glucanotransferase mutant, and after isolation and purification, obtaining the pure enzyme of the Lactobacillus fermentum 2970 4,3-α-glucanotransferase mutant.
